(19) United States
(12) Patent Application Publication
(10) Pub. No.: US 2005/0277618 A1
(43) Pub. Date: Dec. 15, 2005

(54) Methods

(76) Inventors: Stefan Schreiber, Keil (DE); Jochen Hampe, Keil (DE); Monika Stoll, Keil (DE)

Correspondence Address: WHITE & CASE LLP;PATENT DEPARTMENT, 1155 AVENUE OF THE AMERICAS, NEW YORK, NY, 10036, US

(21) Appl. No.: 10/477,507

(22) PCT Filed: May 8, 2002
(86) PCT No.: PCT/GB02/02129

§ 371 Date: May 9, 2005

(30) Foreign Application Priority Data
May 12, 2001 (GB) 0111637.5

(51) Int. Cl. A61K 31/737

(52) U.S. Cl. 514/54

(57) ABSTRACT

The invention provides a method of identifying a compound useful for treatment of IBD which comprises assaying the compound for its ability to modulate the activity or amount of 3OST4. The assay is selected from measurement of 3OST4 activity using a cell line which expresses 3OST4 or using purified 3OST4 protein, and measurement of 3OST4 transcription or translation in a cell line expressing 3OST4. The invention also provides a method of preparing a pharmaceutical composition, a diagnostic method for the determination of susceptibility to IBD, a method for the diagnosis of IBD or a predisposition thereto and use of a compound able to modulate the activity or amount of 3OST4 in preparation of a medicament for the treatment of IBD.

Inflammatory bowel disease (IBD) is characterised by a chronic relapsing intestinal inflammation of the gastrointestinal tract. It affects ˜1/1,000 individuals in Western countries with the median age of onset in early adulthood. To date, the etiology of this disease is unknown. Based on clinical and histopathological features, IBD is categorised into two main subtypes, Crohn's disease (CD) (On Line Mendelian Inheritance in Man—a database produced by Johns Hopkins University available at NCBI, OMIM 266600) and ulcerative colitis (UC) (OMIM 191390). Although the cause of IBD is unknown, both familial clustering of the disease and increased concordance in monozygotic twins shows a strong genetic susceptibility. Estimates of sibling risk (λs) show a range of 10-50, suggesting that genetic factors play a significant role in the predisposition to IBD. Based on epidemiological data, IBD is considered to be a multifactorial disorder that is caused by multiple susceptibility genes as well as environmental factors. In the present context the term IBD is intended to include IBD, as well as Crohn's disease and ulcerative colitis.

Previous genome wide linkage analyses have identified a number of susceptibility locus for IBD, e.g. IBD1 (OMIM 266600) (Hugot et al. 1996, Brant et al. 1998, Curran et al. 1998, Hampe et al. 1999a), IBD 2 (OMIM 601458) (Duerr et al 1988, Parkes et al. 2000), IBD3 (OMIM 604519) (Hampe et al: 1999b), IBD7 (OMIM 605225) (Cho et al. 1998, Cho et al. 2000). The IBD1 locus is located on the pericentromeric region of human chromosome 16, mapped to the region 16 p12-q13.

Heparan sulfate D-glucosaminyl 3-O-sulfotransferase-4 (3OST4) belongs to a group of Heparan sulfate D-glucosaminyl 3-O-sulfotransferase isoenzymes. Five members of this group, 3OST1, 3OST2, 3OST3A, 3OST3B, and 3OST4, have been identified so far. Single copy genes encode 3OST1, 3OST2 and 3OST4, whereas 3OST3A and 3OST3B have suggested to be encoded by at least two genes each. (Shworak et al. 1997, Shworak et al. 1999, WO 99/22005).

Heparan sulfate is a glucosaminoglycan that is synthesised initially as extended repeats of a disaccharide unit containing glucuronic acid in (1-4) linkage to N-acetylglucosamine ([GlcA(1-4)GlcNAc(1-4)]n). These GAG chains undergo a series of ordered modifications, catalysed by the heparin sulfotransferases.

Thus, the 3OST enzymes belong to a family of carbohydrate sulfotransferases of which one group are the heparin sulfotransferases. Modifications of Heparan sulfate (HS) and other glycosaminoglycans (GAG) by carbohydrate sulfotransferases largely determine the biophysical properties of the mature proteoglycan and confer highly specific biological functions to these complex molecules. Such modifications are implicated in a wide variety of biological functions such as cell adhesion, modification of growth factor and chemokine activities, angiogenesis and coagulation. In addition, certain sulfations have been shown to contribute to specific recognition epitopes recognised by viral receptors, thus mediating or contributing to viral entry into host cells (see Bowman and Bertozzi, 1999 and Sasisekharan and Venkataraman, 2000 for review; Shukla et al. 1999).

For example, heparin, a complex GAG synthesised exclusively by mast cells, contains a defined hexasaccharide epitope, which binds to antithrombin and imparts a conformational change that reveals its high-affinity binding site for thrombin, leading to inhibition of this enzyme and therefore stopping the blood-clotting process (Rosenberg and Damus, 1973). The affinity of this antithrombin binding site in heparin depends crucially on 3-O-sulfated N-sulfoglucosamine within the hexasaccharide epitope (Rosenberg et al. 1997). The 3-O-sulfation has been shown to be the final and rate-limiting step in biosynthesis of the antithrombin-binding epitope of heparin and is catalysed by 3OST1 (Shworak et al. 1996, Shworak et al. 1997). The specific and distinct functions of 3OST iso-enzymes is exemplified by the fact that only 3OST1 modifies HS to enable binding to antithrombin (Liu et al. 1999), and only 3OST3 catalyse the 3-O-sulfation of N-acetylglucosamine which is required for HS dependent herpes simplex virus type 1 entry (Shukla et al. 1999).

Growth-factor binding to heparan sulfate proteoglycan is also dependent on particular sulfated epitopes within the GAGs (Faham et al. 1996). Furthermore, several viruses use specific sites on heparan sulfate proteoglycans for binding to cells and/or viral entry. Shukla et al. (1999) recently reported that 3-O-sulfation of specific N-acetylglucosamine residues in heparan sulfate chains, catalysed by 3OST3, is required for heparan-sulfate-dependent herpes simplex virus type 1 entry.

The heparan sulfotransferases utilise 3′-phosphoadenosine 5′-phosphosulfate (PAPS) as an activated-sulfate donor. The substrate specificity of the 3OST isoforms have been investigated by Liu et al. (1999) and these specificities explain the distinct biological effects conferred by the 3OST isoenzymes.

The tissue distribution of 3OST isoenzymes differs. Previous to the data presented herein, expression of 3OST4 had only been found in brain, whereas the 3OST1 and 3OST3 isoenzymes were more widely distributed (Shworak 1999). Furthermore, the expression of 3OST isoenzymes had not been studied in gastrointestinal tissue.

So far, no specific function has been ascribed to HS/GAG modified by 3OST4. The specific involvement of HS or other GAGs in the pathogenesis of IBD is not known. However, the leading hypotheses for pathogenetic mechanisms in these diseases involve host microbial interactions, disturbance of mucosal immune responses and epithelial functions, such as innate immune responses and epithelial barrier function. The fundamental role that sulfated GAGs have in a wide array of biological responses, including immune responses, control of growth and healing processes and cell-cell interactions, point at several possibilities for disrupted GAG related functions to be involved in the etiology of IBD.

The present invention is based on the discovery of a genetic predisposition for IBD associated with genetic markers located in a region on chromosome 16 in close proximity to the location of the 3OST4 gene. The use of compounds able to modulate the activity or amount of 3OST4 has been identified as a new therapeutic concept for the treatment of IBD by the present inventors. The analysis of the sequence of the 3OST4 gene will provide a new method for the diagnosis of susceptibility to IBD.

According to one aspect of the present invention there is provided use of a compound able to modulate the activity or amount of 3OST4 for the treatment IBD. Modulation of the amount of 3OST4 by a compound may be brought about for example through altered gene expression level or message stability. Modulation of the activity of 3OST4 by a compound may be brought about for example through compound binding to 3OST4 protein. In one embodiment, modulation of 3OST4 comprises a compound able to reduce the activity or amount of 3OST4. In another embodiment, modulation of 3OST4 comprises a compound able to increase the activity or amount of 3OST4. An example of a compound able to modulate the activity of 3OST4 is an antibody. Antibodies can be prepared using any suitable method. For example, purified polypeptide may be utilized to prepare specific antibodies. The term “antibodies” is meant to include polycional antibodies, monoclonal antibodies, and the various types of antibody constructs such as for example F(ab′)₂, Fab and single chain Fv. Antibodies are defined to be specifically binding if they bind the allelic variant of SLC10A2 with a Kₐ of greater than or equal to about 10⁷ M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunizations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKearn, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols, see for example “A Practical Guide to ELISA” by D. M. Kemeny, Pergamon Press, Oxford, England.

According to another aspect of the present invention there is provided use of compounds able to modulate the activity or amount of 3OST4 in preparation of a medicament for the treatment of IBD.

According to yet another aspect of the present invention there is provided a method of identifying a compound potentially useful for treatment of IBD which comprises assaying the compound for its ability to modulate the activity or amount of 3OST4. Preferably the assay is selected from:
i) measurement of 3OST4 activity using a cell line which expresses 3OST4 or using purified 3OST4 protein; and
ii) measurement of 3OST4 transcription or translation in a cell line expressing 3OST4.

The assay used to determine the effect of a compound to be tested on the activity of 3OST4 can be based on measurement of ³⁵S labelling of heparin sulfate following incubation of 3OST4 with ³⁵S labelled PAPS (3′phosphoadenosine 5′phosphosulfate) and unlabeled heparin sulfate.

The assay used to determine the effect of a compound to be tested on the transcription or translation of 3OST4 can be based on
i) measurement of the amount of 3OST4 mRNA formed using e.g. Northern blot analysis or quantitative real time PCR,
ii) measurement of the amount of 3OST4 protein formed using e.g. Western blot analysis, or immunochemical analysis such as ELISA, or
iii) measurement of 3OST4 activity as described above,
   in cells expressing 3OST4.

The cells used in the assay can be cells naturally expressing a 3OST4 or transfected cells expressing a recombinant 3OST4. Preferably the 3OST4 is the human recombinant 3OST4.

The 3OST4 may be expressed in a variety of hosts such as bacteria, plant cells, insect cells, fungal cells and human and animal cells. Eukaryotic recombinant host cells are especially preferred. Examples include yeast, mammalian cells including cell lines of human, bovine, porcine, monkey and rodent origin, and insect cells including Drosophila and silkworm derived cell lines. Cell lines derived from mammalian species which may be used and which are commercially available include, L cells L-M(TK-) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

The expression vector comprising a nucleic acid encoding a 3OST4 may be introduced into host cells to express a polypeptide of the present invention via any one of a number of techniques including calcium phosphate transformation, DEAE-dextran transformation, cationic lipid mediated lipofection, electroporation or infection.

The transfected host cells are propagated and cloned, for example by limiting dilution, and analysed to determine the expression level of recombinant 3OST4. Identification of transformed host cells that express 3OST4 may be achieved by several means including immunological reactivity with antibodies and/or the detection of biological activity using the assays described herein.

Recombinant human 3OST1 has been transiently expressed in COS-7 cells under the control of the CMV promoter. The expressed protein, both in the native full length form and a protein-A tagged form, was found to be fully active and exclusively catalysed the transfer of sulfate to the 3-O-position of the glucosamine units of heparin (WO 99/22005).

Transcriptional regulation of gene expression is mediated by specific DNA elements in the promoter that directs binding of transcription factors, which thereby mediate transcription of the gene. Eukaryotic transcription factors can be divided in two main groups i) basal transcription factors that interact with promoter sequences proximal to the start of transcription, thereby initiating transcription upon recruitment of RNA polymerase II and ii) transcription factors that bind to specific distal promoter elements, thereby modulating the transcription upon contact with the basal transcription machinery. A fundamental physiological process in the eukaryotic organism is that cells can communicate with their environment and respond to extracellular stimuli through signalling molecules, such as hormones and growth factors. The final event for such signalling is the binding of transcription factors to specific distal promoter elements leading to for example up-regulated or tissue specific gene expression. Because of their regulatory role, promoter elements are putative targets for screening of therapeutic agents. The sequence of the human 3OST4 promoter, disclosed in this patent application, makes it possible to screen for therapeutic agents selectively regulating transcription of 3OST4.

Suitable host cells are cells known to express 3OST4 or cells known to express transcription factors that can influence the transcription of 3OST4. Host cells transfected with DNA encoding specific transcription factors can preferably be used to study the interaction with defined transcription factors and the 3OST4 promoter.

The assay used to determine the effect of a compound to be tested on the transcription of 3OST4 can be based on measurement of the activity of the 3OST4 promoter using a reporter gene system. The reporter gene system is an expression system comprising nucleic acid molecules constituting an 3OST4 promoter, or fragments thereof, the expression system further comprising a reporter gene, the promoter and the reporter gene being positioned so that the expression of the reporter gene is regulated by the 3OST4 promoter. The amount of reporter protein formed is used as an indication of the activity of the 3OST4 promoter.

Preferably the 3OST4 promoter is the human 3OST4 promoter, preferably the nucleotide sequence 1-10779 of SEQ ID NO: 3, or fragments thereof.

Suitable reporter genes that can be used for the construction of the reporter gene system are e.g. the firefly luciferase gene, the bacterial chloramphenicol acetyl transferase (CAT) gene, the β-galactosidase (β-GAL) gene, and the green fluorescent protein (GFP).

According to another aspect of the present invention there is provided a method of preparing a pharmaceutical composition which comprises:
i) identifying a compound as useful for treatment of IBD according to a method as described herein; and
ii) mixing the compound or a pharmaceutically acceptable salt thereof with a pharmaceutically acceptable excipient or diluent.

In yet another aspect of the present invention there is provided a diagnostic method comprising the analysis of the sequence of chromosome 16, or a part thereof, in a DNA sample obtained from a patient, for the determination of susceptibility to IBD, further comprising determining the presence or absence of an allele of a polymorphic marker located on a chromosomal fragment corresponding to the BAC 504N19 (EMBL AF265340), BAC A-249B10 (EMBL AC002288) or BAC RP11-451N20 (EMBL AC060785). Preferably, the method comprises the analysis of the sequence of the 3OST4 gene, or a part thereof, in a DNA sample obtained from a patient, for the determination of susceptibility to IBD, and further comprises determining the presence or absence of an allele of a polymorphic marker located in the 3OST4 gene of the patient.

In another aspect of the invention there is provided a method for the modulation of a immune response to auto-immune epitope on the 3OST4 protein. Preferably this method comprises a method for the induction of immunologic tolerance. Immunologic tolerance can be induced e.g. by oral immunisation using the B subunits of cholera toxin (CT) from V. cholera or the heat labile toxin (CT) from E. coli in conjugation with the auto-antigen (Simmons et al. 2001).

Autoimmune responses are known to be a cause of a number of inflammatory diseases, such as rheumatoid arthritis, pancreatitis and multiple sclerosis.

In another aspect of the invention is there is provided a method for the diagnosis of IBD comprising the measurement auto-antibodies directed against the 3OST4 protein.

Abbreviations

| | |
|---|---|
| Heparan sulfate D-glucosaminyl 3-O- | 3OST4 |
| sulfotransferase-4 | |
| Affected sibling pair | ASP |
| Bacterial artificial chromosome | BAC |
| chloramphenicol acetyl transferase | CAT |
| Crohn's disease | CD |
| Centimorgan | CM |
| cholera toxin | CT |
| Glycosaminoglycans | GAG |
| green fluorescent protein | GFP |
| Heparan sulfate | HS |
| Inflammatory bowel disease | IBD |
| heat labile toxin | LT |
| On Line Mendelian Inheritance in Man | OMIM |
| 3′-phosphoadenosine 5′-phosphosulfate | PAPS |
| Pedigree disequilibrium test | PDT |
| Sequence tagged site | STS |
| Transmission disequilibrium test | TDT |
| ulcerative colitis | UC |

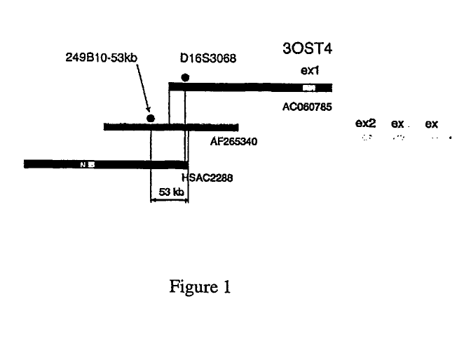

The invention will now be illustrated with reference to the following non-limiting Examples in which FIG. 1 illustrates a map of the chromosomal region studied.

AMPLITAQ™ available from Perkin-Elmer Cetus, is used as the source of thermostable DNA polymerase.

General molecular biology procedures can be followed from any of the methods described in “Molecular Cloning—A Laboratory Manual” Second Edition, Sambrook, Fritsch and Maniatis (Cold Spring Harbor Laboratory, 1989) or in “Current Protocols in Molecular Biology Volumes 1-3, edited by F M Asubel, R Brent and R E Kingston; published by John Wiley, 1998.

### EXAMPLE 1

We have narrowed the region containing the susceptibility gene for IBD1 to ˜7 Mb in physical distance using high-density genotyping with STS markers and yet unpublished single nucleotide polymorphisms, followed by standard procedures for linkage disequilibrium testing (Terwilliger, 1995). TDT-testing across the region flanked by STS markers, D16S401 and D16S409, respectively, with a total of 32 markers (13 STS and 19 SNPs) verified the initial linkage data and revealed linkage distortion at two sites. Our data indicate that the susceptibility gene for IBD1 is very likely located in the region flanked by D16S401 and D16S409.

1.1 Family Ascertainment and Phenotypes:

Patients were recruited from IBD programs at Charité University Hospital (Berlin) and the Ist Department of Medicine at the Christian-Albrechts-University of Kiel. Kindreds with ≧2 affected siblings diagnosed with IBD were sampled, leading to a more than 250 affected sib pairs included in the analysis. Written informed consent was obtained from all study participants. Recruitment protocols were approved by institutional review committees. The diagnosis of IBD and classification in CD and UC were determined by standard diagnostic criteria as described by Lennard-Jones (1989) and Podolsky (1991). Patients were directly examined by one or more of the principal investigators. Alternatively, two written records, containing a detailed disease history and results of all diagnostic procedures, were obtained for each patient and reviewed by the principal investigators. A venous blood sample was obtained from the affected siblings and their parents, if possible.

1.2 Genotyping:

Genomic DNA was prepared from whole blood samples with the Puregene system (Gentra Systems). The individual DNA samples were arrayed on 96-well microtiter plates and subjected to amplification by PCR, with individual marker amplicons. Microsatellite markers were genotyped with fluorescent methods, as described by Hall and Nanthakumar (1997). Data were collected using ABI377 automated sequencers and data analysis was performed using GENESCAN (Vs. 2.1) and GENOTYPER (Vs. 1.1.1). Allele analysis and individual allele calling were performed as described by Hall and Nanthakumar (1997) and Idury and Cardon (1997). SNPs were identified by genomic sequencing using ABI3700 automated sequencers, cDNA selection and mutation detection methodologies. Genotyping of informative SNPs was performed by real-time PCR using the TAQMAN™ technology from PE Biosystems. PCR reactions were performed in an ABI9700, and fluorescence results were determined with the use of ABI7700 sequence-detector single-point measurement. Allele analysis and individual allele calling were performed as described by Hall and Nanthakumar (1997). Genetic maps were constructed with the automated mapping program MULTIMAP (Vs. 2.0, Matise et al 1994). The resulting distances between markers, determined by the Kosambi map algorithm, are given in centimorgans (cM).

1.3 Genetic Analysis

Genetic analyses were conducted with the use of the two aforementioned standard diagnostic categories, CD and UC. A third category, ALL, contains CD/CD, UC/UC and CD/UC (mixed ASPs) and, therefore, represents IBD as a single phenotype for analysis. Allele frequencies for each marker were calculated from the cohort genotyping data for all individuals. Association statistics were calculated by the TDTLIKE program from the ANALYZE software package (Terwilliger 1995). Tested alleles are restricted to ≧10 observed transmissions. The program algorithm provides p-values corrected for the testing of multiple alleles.

Sequence Information for Markers (1STS, 3 SNPS) in the Region Between D16S401 and D16S409

| | | |
|---|---|---|
| D16S3068 | EMBL SSA34SZNS | |
| A-249B10_27kb | TTCCTGTTTATTTATTATcATTTTATGGAGAGTGTTGGTACAGAAA | SEQ ID NO 5 |
| | TTCCTGTTTATTTATTATgATTTTATGGAGAGTGTTGGTACAGAAA | SEQ ID NO 6 |
| A-249B10_37kb | TGGGAAAGACTTCAATAaGTTTACATTCCTAGATTCTCC | SEQ ID NO 7 |
| | TGGGAAAGACTTCAATAtGTTTACATTCCTAGATTCTCC | SEQ ID NO 8 |
| A-249B10_53kb | ACTTATTGAGGcACCCAGTTGACACCCATG | SEQ ID NO 9 |
| | ACTTATTGAGGtACCCAGTTGACACCCATG | SEQ ID NO 10 |

TDT-Results for Positive Markers on 16p in 200 ASPs for IBD

a) TDTLIKE (Terwilliger, 1995)

| | | | TDT | p-value | | |
|---|---|---|---|---|---|---|
| | | Con- | single | single | TDT | p-value |
| Marker | Cases | trols | point | point | multipoint | multipoint |
| D16S3068 | 103 | 52 | 16.78 | 0.00015 | 12.1294 | 0.00025 |
| (allele G) | | | | | | |
| A-249B10_27kb | 106 | 75 | 5.309 | 0.02532 | 3.94 | 0.0234 |
| (allele B) | | | | | | |
| A-249B10_37kb | 103 | 69 | 6.72 | 0.01160 | 5.37 | 0.0102 |
| (allele B) | | | | | | |
| A-249B10_53kb | 108 | 72 | 7.20 | 0.00880 | 7.2 | 0.0077 |
| (allele A) | | | | | | |

Allele G is a STS (CA)ₙ repeat
Alleles A & B represent one of the alleles at the SNP positions mentioned above.

b) PDT (Martin et al. 2000)

| Marker | Z-score | Global χ² | P-value |
|---|---|---|---|
| D16S3068 | 3.241 | 21.205 | 0.0118 |
| A-249B10_27kb | 3.019 | 9.116 | 0.0025 |
| A-249B10_37kb | 2.782 | 7.737 | 0.0054 |
| A-249B10_53kb | 3.326 | 10.475 | 0.0012 |

The strongest association was seen for the D16S3068 marker.
1.4 Bioinformatics

Three human BACs from chromosome 16, RP11-451N20 (EMBL AC060785), 504N19 (EMBL AF265340), 249B10 (EMBL AC002288) comprising the marker D16S3068 were analyzed for potential disease related genes. Parts of the sequence corresponding to the 3OST4 cDNA (EMBL AF105378) sequence were found on the BAC RP11-451N20 (EMBL AC060785). The sequence of this BAC, comprising 14 unordered fragments, were further analysed and found to contain exon 1 and exon 2 of the 3OST4 gene. Furthermore, a 10 kbp fragment comprising 3OST4 promoter sequences was identified on one of these fragments.

1.5 Expression Analysis

Expression of 3OST4 is determined by real-time PCR using the TAQMAN™ technology (PE Biosystems). A panel of mRNA prepared from different human tissues as well as mRNA prepared from colon biopsies of IBD patients are used to prepare cDNA samples. Primers that specifically amplify a fragment of the 3′ untranslated part of the 3OST4 cDNA but which not bind to the cDNA of the other highly homologous 3OST iso-enzymes are designed.

Using the above-mentioned technologies, 3OST4 mRNA was detected in material prepared from human intestinal tissue from IBD patients and controls. 3OST4 mRNA was also detected in commercially available samples (cDNA panels) derived from human gastro-intestinal tissues. Furthermore, the presence of 3OST4 mRNA was detected in human intestinal cell lines. In these cell lines it was found that inflammatory stimuli down-regulates 3OST4 mRNA (see table below).

3OST4 mRNA Detection (+low to ++++ High)

| | Uninflamed State | Inflamed State |
|---|---|---|
| Intestinal tissue | + | + |
| Gastrointestinal tissue | + | N/a |
| (cDNA panel) | | |
| Intestinal cell lines | ++ | + |

1.6 Cloning and Sequencing of the Complete 3OST4 Gene

BACs are screened by PCR analysis using primers derived from the 3OST4 cDNA sequence. Positive BACs are sequenced and anlalysed for the presence of 3OST4 gene sequences. The exon/intron structure of the 3OST4 gene is defined and promoter sequences, proximal and distal, are identified.

1.7 Identification of SNPs in the 3OST4 Gene

PCR primers are constructed based on the DNA sequences flanking the exons as well as the promoter region of the 3OST4 gene. After PCR amplification, the exons and the promoter region are sequenced from genomic DNA samples obtained from 23 IBD affected individuals as well as 20 normal controls. Sequence variants present in more than 10% of the individuals are identified and selected for SNP genotyping in the rest of the cohort using the TAQMAN™ technology described previously.

1.8 Method of Diagnosing Susceptibility to IBD in a Patient

DNA samples are obtained from patients. The presence or absence of an allele of one or more polymorphic marker, which is associated with increased susceptibility to IBD, is determined, e.g. using the TAQMAN™ technology.

1.9 3OST4 Assays

The assay used to determine the effect of a compound to be tested on the activity of 3OST4 is based on measurement of ³⁵S labelling of Heparin sulfate following incubation of 3OST4 with ³⁵S labelled PAPS (3′phosphadenosine 5′phosphosulfate) and unlabeled heparin sulfate (HS).

HS sulfotransferase activity is determined in 200 nM unlabeled HS, 60×10⁶ cpm of ³⁵S PAPS, 50 mM MES (Sigma), pH 7.0, 10 mM MnCl2, 5 mM MgCl2, 75 μg/ml protamine chloride (Sigma), 120 μg/ml bovine serum albumin, and 1% Triton X-100 (v/v) in a final volume of 50 μl. The reaction mixture is incubated at 37° C. for 2 h, quenched by heating to 100° C. for 2 min and spun at 14,000×g for 2 min to remove insoluble material. The sample is then subjected to a 200-μl DEAE-Sepharose column to isolate ³⁵S HS.

Heparan sulfotransferase activity is defined as the amount of transfer of sulfate from PAPS to HS under standard assay conditions

### REFERENCES

Bowman K G and Bertozzi C R. 1999. Carbohydrate sulfotransferases: mediators of extracellular communication. Chemistry & Biology 1999, 6:R9-R22.
Brant S R. 1998. American families with Crohn's disease have strong evidence for linkage to chromosome 16 but not chromosome 12. Gastroenterlogy 115: 1056-1061.
Cho J H et al. 1998. Identification of novel susceptibility loci for inflammatory bowel disease on chromosomes 1p, 3q, and 4q: evidence for epistasis between 1p and IBD1. Proc. Nat. Acad. Sci. 95: 7502-7507.
Cho J H et al. 2000. Linkage and linkage disequilibrium in chromosome band 1p36 in American Chaldeans with inflammatory bowel disease. Hum. Molec. Genet. 9: 1425-1432.
Curran M E et al. 1998. Genetic analysis of inflammatory bowel disease in a large European cohort supports linkage to chromosomes 12 and 16. Gastroenterology 115:1066-1071
Duerr R H et al. 1998. Linkage and association between inflammatory bowel disease and a locus on chromosome 12. Am. J. Hum. Genet 63: 95-100.
Faham S et al. 1996. Heparin structure and interactions with basic fibroblast growth factor. Science 271, 1116-1120.
Hall J and Nanthakumar E. 1997. Automated flourescent geno-typing. In: Boyle A L (ed) Current protocols in human genetics. Vol 2. John Wiley & Sons, pp 2.8.1-2.8.19
Hampe J et al. 1999a A genomewide analysis provides evidence for novel linkages in Inflammatory Bowel Disease in a large European cohort. Am. J. Hum. Genet. 64:808-816.1
Hampe J et al. 1999b. Linkage of inflammatory bowel disease to human chromosome 6p. Am. J. Hum. Genet 65: 1647-1655.
Hugot J P et al. 1996. Mapping of a susceptibility locus for Crohn's disease on chromosome 16. Nature 379:821-823
Idury R M and Cardon L R. 1997. A simple method for automated allele binning in microsatellite markers. Genome Res 7:1104-1109
Lennard-Jones J E. 1989. Classification of inflammatory bowel disease. Scand J Gastroenterol Suppl 170:2-6
Liu J et al. 1999. Expression of Heparan Sulfate D-Glucosaminyl 3-O-Sulfotransferase isoforms reveals novel substrate specificities. JBC, 274, 5185-5192.
Martin E R et al. 2000. A test for linkage and association in general pedigrees: the pedigree disequilibrium test. Am J Hum Genet 67:146-154
Matise T C et al. 1994. Automated con-struction of genetic linkage maps using an expert system (MultiMap): a human genome linkage map. Nat Genet 6:384-390
Parkes M et al. 2000. The IBD2 locus shows linkage heterogeneity between ulcerative colitis and Crohn disease. Am. J. Hum. Genet. 67: 1605-1610.
Podolsky D K. 1991. Inflammatory bowel disease. N Engl J Med 325:928-937
Rosenberg R D and Damus P S. 1973. The purification and mechanism of action of human antithrombin-heparin cofactor. J. Biol. Chem. 248, 6490-6505
Rosenberg R D et al. 1997. Heparan sulfate proteoglycans of the cardiovascular system; specific structures emerge but how is synthesis regulated?. J. Clin. Invest. 99, 2062-2070.
Terwilliger J. 1995. A powerful likelihood method for the analysis of linkage disequilibrium between trait loci and one or more polymorphic marker loci. Am J Hum Genet 56:777-787 Sasisekharan R and Venkataraman G. 2000. Heparin and heparan sulfate: biosynthesis, structure and function. Current Opinion in Chemical Biology 4:626-631
Shukla D et al. 1999. A novel role for 3-O-sulfated heparan sulfate in herpes simplex virus 1 entry. Cell 99, 13-22.
Shworak N W et al. 1996. Cell-free synthesis of anticoagulant heparan sulfate reveals a limiting converting activity that modifies an excess precursor pool. J. Biol. Chem. 271, 27063-27071.
Shworak N W et al. 1997. Molecular cloning and expression of mouse and human cDNAs encoding Heparan Sulfate D-Glucosaminy 13-O-Sulfotransferase. JBC 272, 28008-28019.
Shworak N W et al. 1999. Multiple isoforms of Heparan Sulfate D-Glucosaminyl 3-O-Sulfotransferase. JBC 274, 5170-5184.
Simmons C P et al. 2001. Immunomodulation using bacterial enterotoxins. Scand J Immunol 53 (3), 218-226.

1. A method of identifying a compound useful for treatment of IBD which comprises assaying the compound for its ability to modulate the activity or amount of 3OST4.

2. The method according to claim 1 wherein the assay is selected from:
i) measurement of 3OST4 activity using a cell line which expresses 3OST4 or using purified 3OST4 protein; and
ii) measurement of 3OST4 transcription or translation in a cell line expressing 3OST4.

3. The method according to claim 2 wherein the activity of 3OST4 is based on measurement of ³⁵S labelling of heparin sulfate following incubation of 3OST4 with ³⁵S-labelled 3′phosphoadenosine 5′phosphosulfate and unlabeled heparin sulfate.

4. The method according to claim 2 wherein the assay used to measure the transcription or translation of 3OST4 is selected from any of the following:
i) measurement of the amount of 3OST4 mRNA;
ii) measurement of the amount of 3OST4 protein; or
iii) measurement of 3OST4 activity in cells expressing 3OST4.

5. The method according to any preceding claim wherein the 3OST4 is human recombinant 3OST4.

6. The method according to any one of claims 1-4 which measures the activity of the 3OST4 promoter using a reporter gene system.

7. The method according to claim 6 wherein the 3OST4 promoter is the human 3OST4 promoter.

8. A method of preparing a pharmaceutical composition which comprises:
i) identifying a compound as useful for treatment of IBD according to the method as defined in claim 1; and
ii) mixing the compound or a pharmaceutically acceptable salt thereof with a pharmaceutically acceptable excipient or diluent.

9. A diagnostic method for the determination of susceptibility to IBD comprising the analysis of a DNA sample obtained from a patient for the presence or absence of an allele of a polymorphic marker located on a chromosomal fragment corresponding to BAC 504N19 (EMBL AF265340), BAC A-249B10 (EMBL AC002288) or BAC RP11-451N20 (EMBL AC060785).

10. A method for the diagnosis of IBD or a predisposition thereto comprising the measurement of auto-antibodies directed against 3OST4.

11. A method for the treatment of IBD which comprises administering to a patient in need of such treatment an effective amount of a compound able to modulate the activity or amount of 3OST4.


SEQUENCE LISTING (10 sequences)

SEQ ID NO: 1 - DNA, 3108 bp, Homo sapiens
  CDS (297)..(1667)
gtccgcgcgcccccctcggtccccttgcctgaggctgaggggggggcggtggtgggggggccactcggactcggcgggcagcgtggggcggggggccatgcggccgggctcccccctggcgcagcgggacagcggccagggccgggggcgcagcggcgtcgcttcatgcagccggggcggctgggcagcggcggcggcggcggcggcggcggcggcgggggcggcggctgaaaccatgtccgggcagcgccgggggctgccgccgccgccgccgccgccgcgagccgggagccgcgatggcccggtggcccgcacctcctccgcctccgcctccgcctccacctctggccgcgccgccgccgcccggcgcctctgctaaggggccgccggcgcgcaagctgctttttatgtgcaccttgtccctgtctgtcacctacctgtgctacagcctcctgggcggctcgggctccctgcaattccctctggcgctgcaggagtcgccgggcgccgccgccgagcccccgccgagcccgccgccaccctctctgctgcctacccccgtgcgcctcggcgccccctcgcagccgcccgcgccgccgccgctggacaacgcgagccacggggagccgcccgagcccccagagcagccagccgcccccgggaccgacggctgggggctgccgagcggcggcggaggcgcccgggacgcctggctccggaccccgctggcccccagcgagatgatcacggctcagagcgcgctgccggagagggaagcgcaggagtccagcaccaccgacgaggatctcgcaggccggagagcggccaacgggagcagcgagaggggcggcgccgtcagcacccccgactatggggagaagaagctgccacaggcgctcatcatcggggtcaagaaaggagggacccgcgcgctgctggaggcgatccgcgtgcacccggacgtgcgggcggtgggcgtagagccgcacttcttcgacaggaactacgaaaaggggttggagtggtacagaaatgtgatgcccaagactttggatgggcaaataaccatggagaagactccaagttactttgtgacaaatgaggctcccaagcgcattcactccatggccaaggacatcaaactgattgtggtggtgagaaaccccgtgaccagggccatctctgactacacgcagacactgtcaaagaaacccgagatccccacctttgaggtgctggccttcaaaaaccggaccctcgggctgatcgatgcttcctggagtgccattcgaatagggatctatgcgctgcatctggaaaactggctccagtatttccccctctcccagatcctctttgtcagtggtgagcgactcattgtggaccccgccggggaaatggccaaagtacaggattttctaggcctcaaacgtgttgtgactaagaagcatttctatttcaacaaaaccaaggggttcccttgcctaaagaagccagaagacagcagtgccccgaggtgcttaggcaagagcaaaggtcggactcatcctcgcattgacccagatgtcatccacagactgaggaaattctacaaacccttcaacttgatgttttaccaaatgactggtcaagattttcagtgggaacaggaagagggtgataaatgaggctagagaggcagaggaaggctagtcaataagctaaggaggctccttgcctgagtccttgaataccccagcttctgcagcttcacttgctggagtgccaagtagatctcctcctccttcatgcagccaggattgcctccagtgctgttagcttaggcaaacaggtggatcccatggcatccccatggaggaaccaggcccatctgggcagcagcatctggttgaccagatggccaccagaacccactgttcattcttatcttctgctagttaatatagcctgaagacagaggataaatagttgtcaatgtcagagacagtgctattaatgtatatgtgagcgacaaaaaaggtctgctttataggggttctcactctagcttggggagcccagggttctagccctgtatctgtcatgggcacctgctgtctaaacctctgcttgggcttctccccagaatgcactttgtggctgagtgctccaggactcctagggagcaagctcctccctctaaggtgtttctagtcttctctttaaaggtctcatcccacaacccctgacttcctccctccccacatcatgaaggcagaggcatgcacattcctcactgaaaaagaaaacacacacccacccacacacacacacacagaagaaaatgaaagctgacacacctcgaagccttctttccaagagccctctaaatggggttgggtctcactcttcatgagtatcctgggttgtgcagaagcttagcatatgcccttgtgttcggatcaggcccacagggctgctcaaagagtagagtaattgtaaccgaggtcagagctctggggttggcagagatgagtggccatatctgggggtaaaagaagaaatcctgtcctcttggtgggaggttaccttacctgaagaccatctctcccaagcactgtagttctgagcatgtttttggggtggactctgtcccctagggtccctagaagggcaaagaccagagagttgacaagtctgttattaggaataatccttagccatgtaatggagaaaggagcagtcagcattcttccaatttgccccaccaccacctcctcgggcttcattttctctatttagagatggcagagagtgaggtagtggcgagaaagctgactccattcatcagatccagtttatgagggttgggggtgagcaagggctgtctgcagaaacccccatcaagagctgctgaatgaagtgtcccttcccatcagtttgattcaattaaaatgcatcatttgacataaagcacttgttcacagatctccaaaaccaggaattgttctagtaaaactggaaatttgtatgagtggggggagttaaatctgttcagctgttattaaactgtcatttctcccgctaaatgaaaaccgtgttgttataaagcttaatgcaacctgatta

SEQ ID NO: 2 - protein, 456 aa, Homo sapiens
MARWPAPPPPPPPPPPLAAPPPPGASAKGPPARKLLFMCTLSLSVTYLCYSLLGGSGSLQFPLALQESPGAAAEPPPSPPPPSLLPTPVRLGAPSQPPAPPPLDNASHGEPPEPPEQPAAPGTDGWGLPSGGGGARDAWLRTPLAPSEMITAQSALPEREAQESSTTDEDLAGRRAANGSSERGGAVSTPDYGEKKLPQALIIGVKKGGTRALLEAIRVHPDVRAVGVEPHFFDRNYEKGLEWYRNVMPKTLDGQITMEKTPSYFVTNEAPKRIHSMAKDIKLIVVVRNPVTRAISDYTQTLSKKPEIPTFEVLAFKNRTLGLIDASWSAIRIGIYALHLENWLQYFPLSQILFVSGERLIVDPAGEMAKVQDFLGLKRVVTKKHFYFNKTKGFPCLKKPEDSSAPRCLGKSKGRTHPRIDPDVIHRLRKFYKPFNLMFYQMTGQDFQWEQEEGDK

SEQ ID NO: 3 - DNA, 12277 bp, Homo sapiens
  exon (10780)..(11811)
  CDS (11078)..(11809)
  promoter (1)..(10779)
  intron (11812)..(12277)
ttagccgggcatggtggcgggtgcttgtagtcacaggctgctcgggaggttgaggcaggagaatggcatgaaccctggaggcagagcttgcagtgagccgagattgcgccactgcactccagcctgggcgacagagcgagactccatctcaaaaaaaaaaaagatgtggagagctggtttgttctctttaactgcagaatcacattacaatatatgaataaacttcagcaatccatttccttactgatggaacaattgtttcccgttatactatgcaaagcaacactctacatacgtcttgtgtttagaagtgagcattttttagtgaagacaccaggaagtggtgtttggtcgtacaatgacccatgtttactttgacctgatactgttacaggtatttcttctccagtgcagttgtgccaatttacattcctttcagaaatatgtgatattattttcctctacattcttgctaagatttgaagctaaaagacattacattttgccactctaaagagcatgacatggaatatcacctgtgttttaattttcacttttctgattaatagcgaggttgaatataatctcttatgattatttgccatccagattttctccttggtgaactgcctatttatatccttttcattattttagtgattttcaagttatttgttggattttttttttgtatatattctgttttgctttggttttttgtttgtttgctttttgagacggagtcttgctctgtcccccaggctggagtgcagtggcgctatctcggctcactgcaacctccacctcccaggttcaagcgattctcctacctcagccttgcaagtagctgggactataggcacctgccaccacacccggctaattgttgtattttttttttttttagtagagacggggtttcaccatgttggccaggctggttttgaactcctgacctcaagtgatccgtccaccttggcctcccaaagtgctgggattacaggtgtgagccactgagcccagccccttttgtatatattctgaatattaaggcattctcccaggttattttttctttcatccttttaatggcatcctttgattaaaataaatttaaaacataggccaattcagtctttgtcagtcttttcctctatgacatatgctttttgttttctgtttatgacactcctacctgaatgttatagggataatatcctatgttttcttacaaaaccgaaaaagcttttcttttcttttttcttttcttttcttttcttttcttttcttttcttttcttttcttttcttttcttttcttttcttttcttttcttttcttttcagagagggtcttttctgttgctcaggccggactgcagtggtgcaattatggctcactgcagcctcgacctcctgggctgaagtgatcatcccatcccagcctaagtggctgggattgcagacccaagccaccatacccatttattttttatagagacagggtacccctatgttgcccaggctggtcttgaacccctgggcctaaacaattctcttgttttggcctcccaaagtgctgggattacaggtgtgagccactgcacccacccataagcttttcttttcatagttatgtctttaatctgtctagattttcttttctgtctggtggcagggggaaactggttttctcttcttgcttatgatttgcctaatgccttatttaataaatagctcattttttttctcacttataatacctccagtacctgtgagtcggaccctatttagaaatagggtcattgtagatgaaagcaagttaggataagattgttaggttatgaccttgcccactacgacaggtgaccttacaagaagacaaaggcaggtgcctgtaatctcagctacttgggaggctgaggcaggagaattgcttgaacctgggaggcagaggttgcagtgagctgagatcgcgccactgcactccagcgtgggtgacagagtgagactcagtctcaaaaaaaaaaaaaagaaaaagaaaaatagaggcccacaggaaaaatgacctgtgatgacagagacagaaaccggagtgacagagctgcaagccaaggaacaccaagaattggccaccaccagaagctaggaagaggcaaagaaggattttccccagagtcacagaggcagcatatcctactgacaccttgatttcagacatctagcttccagaactgtatgacaataaatttctgttgttaaataagtattagtttgcttgccatttattgtttatttctaagtacttattggaatttattgtagttctgaataagtcttttacagttatattttctttacagttatatttattcttttacagttatattttcaagttgtatcctgattgtatatagaaatgcagttactctttcaattttgaatttttgtttttaatgttttttggtagaaatggggtcttgtaatgttgcccaggctggtcttgaactcctggcctcaagtgatcctcctgcttcagcctcccaaagtgctgggattacaggtgtgagcctctgcacttgacctgtattttgaattttattcaccagctttgccaagcttttttgttggtagattttcttttgccaataaacatatagttggcaaataataatctgtttatttcttcccaactctcatgtccttattatgttttcttatcttactgcattaactgcagatgccagtatactgatgaatagaagctacattggaaggcacacttgcattgttccttccattaaatggtttggttttaatatttcatcacgttttatcataatgttaactgtagtttttggcaaatacttgtttttctcttagggaagttgccttcaatttaagttgctaagtgtatgtataatgagtgaaatggatggatgttgcacatactaaaaataatgtccttctttctttacctggttaatccaattttttctttatactgcatatagacatacatgtcccttcctctgtgaagggttctctcatgcctgtactctaggttaggtgctttaacaatgtgcttcctttacactgttggtgggactgtaaactagttaaactattgtggaagatagtgtggcaattcctcaagtatctagaactagtaataccatttgacccagccatcccattactgggtatatacccaaaggattataaatcatgctgctgtaaagacacatgcggccaggtgcggtggctcatgcctgtaatcccagcactttgggaggccgaggtgggtggatcacgaggtcaagagatcgagactaccctggccaacatggtgaaaccccatctctactaaaaatacaaaaacttagccggatgtggtagcaggtgcctgtagtcccagctactcgggaggctgaggaggagaatggcatgaacctgggaggtggagcttgcagtgagccaaaatcgtgccactgcattccagcctgggtgacagagcgagactccgtctcaaaaaaaaaaaaaaaaaaaacgacacatgcacacatatgtttattgcagcactattcataatagcaaagacttggaaccaatccaaatgtccattgatgatggactagattaagaaaatgtggcacatatacaccatggaatactatgcagccataaaaaaggatgacttcatgtcctttgtagggacatggatgaagctggaaaccatcattctgagcaaacactgcatgttctcactcataggtgggaattgaacaatgagaacacttggacacagggtggggaacaccacacaccggggcctgttgtggggtggggggaggggggagggatagcattaggagaaatacctaatgtaaatgacgagttaatgggtgcagcacaccaacatggcacatgtatacgtacgtaacaaacctgcacgttgtgcacatgtaccctagaacttaaagtatgtattaaaaaatgtgcttccacagcagggcattctgtattctctctctctctcttgttttagagatggcatctttctctgtcacccaggttggaatgcaatggtgtgatcatagctcactgcagcctccaactcctgggctcaagtgatccttctgcctcaaactcttgataagctaggactacagaggtgcaccactgcaccaggctaattttttaaatattttgtagagatggtggtcttactatgttgctcaggctggtctgaaactcctggcctcaagtgatcttctcacctccacctcccagagcaccgggattacaggcgtgagccaccatgcctaactgtattttctctcttttagtacttgtctcactttattaaactttaatgaggacagagaccttgctgtctgtctcactactttatcgtcaatgtctaatccagggccaggaatatactagtcgcttaaaaatgacaattgaaagaatgaataaataagtgaaacaaaagaagagcaagaggtttataaaactgaacaaagcagcattagacgtcaaaacactaatccagttccaggtttgtgaagggtttatgtcaatgaaattacagaaggtccaataagataaaatatttgaattaatggagattggccccctatatttctagatgggaaaattccaacattatggatttattatgacattggagtttcaaactgttgagaaataaccagtgctaggaaaaaaaagaaagagttagatagcttggtaaaactatacacaaacatgaatttcagatggtttaaacatttgtgtgtagaaaataaacaaaaaatgtcctagaagaaagtatagatgaatgtttacacagtttgagggatgcctttttccatcaaaggtaaaatacacaaaaaacatcactgagacattgactacataataatgaaaaacatatgtcatcagaaacactaaaaccagttaattggtaaaggcaaattggtaagtcaataattggaagatatctgttagaaaaaaaaaccagatacctctaataaaaaaagacttttttgaatcaacgagctagcaaccaagcaatagaaaacttataaaaatacaataaacaggcaaataaacataaaacatgaaagataaatgtcagatcaagcggctgtaacaaaatataatagattaagtggcttaaaaaaaggaaatttcttttcttttttttttttagacggagtcttgctctgtcccccaggctggagtgcagtggcactatcttggctcactgcaacctccgtgtcctgggttaaagtgattctctggccttaggctccagagtagctgggacctcagacgcgtgccaccatgcccagctaattttttgtatttttagtagagactgggtttcaccgtgttagccaggatgttcttgatctcctgacctcatgatccgcccacgtcggcctcccaaactgctaggattacgggcatgagccactgcgcccagcctaggaaatttattttctcacagttctgaaaaccggaagtccaagatcaggtgtccagcatggtccagttctggcacaggctctctttttgtcttgcagatggcaaccttcttactgtgtcttcacatggagagggagaaagagagggagtggaatagagatcatttcttcttctccagctacagtcctatctaattaggattctatatgtatgacctcatttaaccttaatcacctcttaaagaccctgttgtccgatacagtcacattggaggttagggcttcagcattttgaatttggagacagggggagggtggcacaattcagtccataacaagaaataaacagccactaaatataaacataagcttggctcttctctttattatttccttacttcctgactcttgctctttttttaacttctcgagttgaacatgtagctcatttgttttaaaattttgtttcggggggataagtatatttgaatctataatctttcttatgagtattgctcaattctatccaccagattttgtcctgttgtgctttctgctctaagcaatttctatttcctatgtaaacacaaggtcttttcaatgcaaggtcttttcagtgcaaggtcttatatgttcatggctactattattctgttttaaatctttttacaagtatacaatgttgtcctttgggctttgtctggaggcttacctccaaatctttatagatacacttttatttgtttgtttgtttgttttttgtagagatggggtttcactatgttgcccaggctgctcttgaactactgagctcaagggatcttcttgcctcggcctctttaagtgctgggattgctggtgtcagccaccatgcctagcttatagatatactttttaatcaacagatgaaagatgaactgaggaaatagagttgctacatacgagacaagaattctgcttatatttctggaaaacttttcttttttacctgatgggcttttaatctttcatttatctacttttgtgaaggggaattctaatattggaggagtgaagccattgtggccaacagcaaggaaaattaatagcatgtgtgctttaatcagtcggatagtctaggttgtgctgtggtaataaccaagccgtaagtctcaggagctgaaaacagcacagatctatttcttgatcatgtacctgtcatgaggggctggatctgtattatccttaatcaacgtgcgggattgagtagatgggtgctgaaatagtttgcatgtgtgtctctgcccaaatctcatgttgaattgcaattcccagtgttggaggtggggcctggtgggaggtaaatggatcttgggggcagatttctcatgaatagtttaccactacccccttggtaatattctagtgacagtgagtgagttctctgaggtttacatcacctcctgcctcgctctcttgctcctgctctggccatgcgatgtgactgtggccccttcaccttccgccatgattgtaactttcctgaggcctcccagaagccaagcagatggcaacataatggttcctatacagcctgcagaaccatgagccaatcaaacctcttttctttacaaattacccagtctcaggtatttatttatagcagtaagagaaaggcctaatacagaagctaagaccctgtattcaacccttttcttctgcaattgctcgggagggaaaacagaccctggcaaatctcctcctgcctctcaaggcttctgcctggaagggacaaacctcacttccgttcatatttcagtagccagagcagcacatagtcacacctaactttaaggggtcagggaagtatatccctaccagatgtccacaacgagactggacaatatttgacgaatgtcagtaatgaccaccacacactgtactgtcctttccaaagccaattttgctgtttgtttgttgatttttaaaaattccctttagttatttgagttactatgaatccttccagtgattttattttatagaaaataagcaaacaaaaaaccctaaaactttaccaatattccagaaggcattagacttgacattataaatgttacaaacagaacatcttaggacagtgaactttagctgatacactgggatagttttgaagaaagagtacacacatatttttcaagaataaagttttaataggaaaaggaagcagagcatggcataggccaatatttagatttgaataattattggtaggcaccccatgtcgggcagggagccagggatttttgcatattctagctaattcaatttaattctttataaaaacaatttgaggtgagtcttatccataatttatacatgcagaaactgaggctgagggaggtaaaatgacctttccaaaagccacttggctagcaaatgacagctttgctcaaatgctatcatctgatactacggagacagaagagaaggacgcagtgtgtatgtttggacagcatgcactgtttggtttggcaggactgcgagatacaccaaaggtagggggaatcttgagtaccaaggtaaagaggttggagtttattagttaggtagtgagataatggttttttgagtcggggggacattggtagagctaaaaaaacattgcctcagaaacattgatctgctattgttttctaggatgaagggaaatgaggcagagtgaggccaggagaccagttagaggaattctgtgggtgttggcaggggagacagaaaactatcttccatcgagtcttcggatccattgggaatgcctggatgacgtcagagttcgccctgtgtaggtagctcccacttttcattgtaggtttctcaaggacttgctcctagaaaaagcgtggctcaaaagtagataaaaaataggcaactgcctaagtgtgaaatttacaaagttcctctccaaaaaagcccgcctcctccctatcacttgtgggcctgacattttaccaaaggggctctattctttcaagagtttgttattaaagcgtgactatttgaggattggaggcaaaagggatactgagaaatgtccttactagcagtgtcaaggcaagtgacataaatgtgtgggggggcaacttgtatgagcactgtgaaaacggcagcatgttcactctacttctcagctctgactgaggggctcaaagttcaggatctgctgatttttcaacagtaacgtcctctccaaggtgttttttttttttccttttttgggaaagcccccagtttaaactattgcagccagtttacatttcttaatgtcactgtgctggccacattcagagctccatttgccaccatcggttttgatacctttttaccaaaacctttcgaaatttgagagcccatctttagtaaaactgggcatggagcagattcgtttggattgctgagaggggagatagaaaagtttgggtgctaggcaggaactgcaaggaggacctgggccatatgccagacatctagtgcctgggccttgaaagggagactggtcgctgacaaggcaatatctgttgcaacccaggcttcctagatgaccaccttggatcatggctcggagcacagggagggctgggcagtgcttgtgtttctctccgttccagttggccccttcccattgacattacagtaatgcagttgtgtgctgtttgaaaaagcatccctagttacacagaatgatttacaggacaccagactctgcatttcagaggtctccagtgtaccataaaaaatatattataaaagaataatctttatctgaactaaagctgcagtgaaggaaactcgtgtccagctgagagcagcagtgagcttttgttcactcagggaaaagtccgtgttctttatcttatttgattaacattttttttctctttggcactaggtatctcgttaatatttagacattatatacattttctttcaactggttttcctattacgtgatcaaacaaaaccagagatgccagccacagcagccaacaagggaaaagcagccccgtcaggcacccagctgtggctggggggcaaatggtactcactagagccacccccaggaggcacctggcagagctctgtgcagagccagccccggttgcagaaagctgagtttgttggagtgcctcagttgatcactctgtctctttctcccatttccctcacttccctgagcaaaatgcaacaggaagcaaagtctagttgtgaatcttccaaagccttctgatgtttaccatgttcccccaggagagggaggtgaggggtggagatctctctgcaaagaaaatacacttaaaaaatttcagcgagccgatgcacagacacccagcaacccagcttgtctccgcttattaggtgttcagagcgacagtggtcccacactatttcagtccaggaaaccatgaactccgttagtggcaatgcccccgaagaggcgcaggtgtgtgcacctgtgattaagggtgtcgaggaggggcagcctcatctcttgaagcagaaagtgttgtcacctggtgatgggacagagggaaaagctctggggctgggaaacctgggggcttgtgtcaaagctccacccatcaggagcttcaagagaagatggggggcggggggcggtggctggaaagatggaagttgggatgggaaagcggttgtagaaaaggattcactcctggaccgaaggcaggaggatatcccgggcgagagaagggagggtcggggatgggctgagttggagtcccagaggaaaagcggaagcgagagcttcgtcacccgctgtcttccagctcccggtgcgcggcaccggaggcaggcgttgggctttacctctctaaaagtactggggcaaaggaatggagaacacggcgtcccgagctcccaagggaggggagtaaacgaggtggggtggggaacaccccaagtgcgtgcgtgctggggggctggggggcacgatctccgttctcccgggtgccccagccctagcgcacgcctccgctcccccgcccccttcgcaggcgcgcgcgaggcgcaccccccttccctcggcggcgccgggcgcgcgcccggccccctcctcctcccctccgcgcctctcctctctcccggcagaaagttagcagcggggaaggaactctgggctgcaacagcgcgcggcggcggcggcagaggctgaagcagaagccgcggcggagccggggaagcgggggcgctgcagacggagcaggtgccgccggcgggtccgcgcgcccccctcggtccccttgcctgaggctgaggggggggcggtggtgggggggccactcggactcggcgggcagcgtggggcggggggccatgcggccgggctcccccctggcgcagcgggacagcggccagggccgggggcgcagcggcgtcgcttcatgcagccggggcggctgggcagcggcggcggcggcggcggcggcggcggcggcggcggcggcgggggcggcggctgaaaccatgtccgggcagcgccgggggctgccgccgccgccgccgccgccgcgagccgggagccgcgatggcccggtggcccgcacctcctccgcctccgcctccgcctccacctctggccgcgccgccgccgcccggcgcctctgctaaggggccgccggcgcgcaagctgctttttatgtgcaccttgtccctgtctgtcacctacctgtgctacagcctcctgggcggctcgggctccctgcaattccctctggcgctgcaggagtcgccgggcgccgccgccgagcccccgccgagcccgccgccaccctctctgctgcctacccccgtgcgcctcggcgccccctcgcagccgcccgcgccgccgccgctggacaacgcgagccacggggagccgcccgagcccccagagcagccagccgcccccgggaccgacggctgggggctgccgagcggcggcggaggcgcccgggacgcctggctccggaccccgctggcccccagcgagatgatcacggctcagagcgcgctgccggagagggaagcgcaggagtccagcaccaccgacgaggatctcgcaggccggagagcggccaacgggagcagcgagaggggcggcgccgtcagcacccccgactatggggagaagaagctgccacaggcgctcatcatcggggtcaagaaaggagggacccgcgcgctgctggaggcgatccgcgtgcacccggacgtgcgggcggtgggcgtagagccgcacttcttcgacaggaactacgaaaaggggttggagtggtacaggtaggaccctgggctccgcgggctggtggagacgcgtgggggagacgcggaggggaagccgcggctttccacgcccttcgagcatccaggcaccgtcccgagaggcccaagcccccgcgagggctctgcaaaccctggcggcgttgctcagggggatcggctgagagggctggactccagcgaaaggtcactttatttcagggcgaggggaggaggtgtcaccctgccctgcctcccgcgctcctcatccaaggaggtgctgtctgaatctgcccagctccaagcctgggaacccccagccctcctgcctgctgggtgtttccgaaaccaggctcttgcggggttctgggattctgggcagaggactttgaggagtgagacaggatggctaaattgactaaggggatttgaggtcccctggaatctcttaaaatcaccctcaaacgcatttgcgtggctggaattc

SEQ ID NO: 4 - protein, 244 aa, Homo sapiens
MARWPAPPPPPPPPPPLAAPPPPGASAKGPPARKLLFMCTLSLSVTYLCYSLLGGSGSLQFPLALQESPGAAAEPPPSPPPPSLLPTPVRLGAPSQPPAPPPLDNASHGEPPEPPEQPAAPGTDGWGLPSGGGGARDAWLRTPLAPSEMITAQSALPEREAQESSTTDEDLAGRRAANGSSERGGAVSTPDYGEKKLPQALIIGVKKGGTRALLEAIRVHPDVRAVGVEPHFFDRNYEKGLEWY

SEQ ID NO: 5 - DNA, 46 bp, Artificial Sequence
  Synthetic primer A-249B10_27kb allele A
ttcctgtttatttattatcattttatggagagtgttggtacagaaa

SEQ ID NO: 6 - DNA, 46 bp, Artificial Sequence
  Synthetic primer A-249B10_27kb allele B
ttcctgtttatttattatgattttatggagagtgttggtacagaaa

SEQ ID NO: 7 - DNA, 39 bp, Artificial Sequence
  Synthetic primer A-249B10_37kb allele A
tgggaaagacttcaataagtttacattcctagattctcc

SEQ ID NO: 8 - DNA, 39 bp, Artificial Sequence
  Synthetic primer A-249B10_37kb allele B
tgggaaagacttcaatatgtttacattcctagattctcc

SEQ ID NO: 9 - DNA, 30 bp, Artificial Sequence
  Synthetic primer A-249B10_53kb allele A
acttattgaggcacccagttgacacccatg

SEQ ID NO: 10 - DNA, 30 bp, Artificial Sequence
  Synthetic primer A-249B10_53kb allele B
acttattgaggtacccagttgacacccatg